(19) United States
(12) Patent Application Publication
(10) Pub. No.: US 2023/0008223 A1
(43) Pub. Date: Jan. 12, 2023

(54) AEROSOL-SHIELDING ENDOSCOPY MOUTHPIECE

(71) Applicant: National Taiwan University Hospital, Taipei City (TW)

(72) Inventors: Wei-Chih LIAO, Taipei City (TW); Chen-Ling PENG, Taipei City (TW); Hsiao-Yu LIN, Taipei City (TW); Yi-Xin ZHANG JIAN, Taipei City (TW)

(21) Appl. No.: 17/854,727

(22) Filed: Jun. 30, 2022

Related U.S. Application Data
(60) Provisional application No. 63/219,860, filed on Jul. 9, 2021.

(51) Int. Cl. A61B 1/00; A61B 90/00

(52) U.S. Cl. CPC A61B 1/00137; A61B 90/05

(57) ABSTRACT

An aerosol-shielding endoscopy mouthpiece (AeroFree mouthpiece) is disclosed. The aerosol-shielding endoscopy mouthpiece includes a bite block part through which the endoscope passes and a mouth and nose-shielding part. Besides allowing for safe passage of the endoscope and respiration of the examinee, the bite block part includes a cover which is configured to mitigate splashing of aerosol and saliva through the orifice for endoscope passage during the endoscopic procedure. The mouth and nose-shielding part contains the bodily fluids and aerosols from the examinee's mouth and nose and reduces the risk of contamination at the time of removal and disposal.

### BACKGROUND OF THE INVENTION

### 1. Field of the Invention

The invention relates to a mouthpiece; in particular, to an aerosol-shielding endoscopy mouthpiece capable of effectively reducing potential contamination and transmission risks.

### 2. Description of the Prior Art

Currently, mouthpieces used for endoscopy still fail to shield the spread of body fluids and droplet aerosols during the examination process, resulting in the exposure of medical personnel to the risk of infection.

Therefore, there is still an urgent need for a mouthpiece capable which can be applied to all endoscopic examinations performed through the oral cavity to reduce the contamination of body fluids and droplet aerosols of the examinee during the examination process.

### SUMMARY OF THE INVENTION

A preferred embodiment of the invention is an aerosol-shielding endoscopy mouthpiece. In this embodiment, the aerosol-shielding endoscopy mouthpiece includes a bite block part and a mouth and nose-shielding part. The bite block part includes an orifice and a cover. The cover is disposed on the orifice. The cover has at least one cut for the endoscope to pass through and shielding a gap between the endoscope and an edge of the orifice to reduce the spread of aerosols and droplet ejected from the orifice during the examination process. The mouth and nose-shielding part is used for shielding a mouth and a nose of an examinee to block body fluids and aerosols from the mouth and the nose of the examinee and reduce contamination during removal and disposal.

In an embodiment, the mouth and nose-shielding part has a first side facing outside and a second side facing oral cavity, and the first side and the second side are made of different materials respectively.

In an embodiment, the first side is made of water-impermeable material and the second side is made of highly absorbent material.

In an embodiment, an adhesive is disposed on an edge of the mouth and nose-shielding part for adhering to a face of the examinee to prevent splashing and liquid seepage.

In an embodiment, a fixing structure is disposed on two sides of the bite block part, and the fixing structure can cooperate with an elastic band or other fixation around the examinee's head to fix the bite block part to prevent the bite block part from falling off or shifting during the examination process.

Compared to the prior art, the aerosol-shielding endoscopy mouthpiece of the invention can be applied to all endoscopic examinations conducted through the oral cavity, thereby achieving the following specific effects: allowing the safe passage of the endoscope and the breathing of the examinee, reducing the spread of aerosols and droplet ejected from the orifice for the endoscope to pass through during the examination process, blocking body fluids and aerosols from the nose and the mouth of the examinee and reducing contamination during removal and processing; therefore, it can effectively reduce potential contamination and transmission risks to improve the drawbacks of the prior arts.

The advantage and spirit of the invention may be understood by the following detailed descriptions together with the appended drawings.

### BRIEF DESCRIPTION OF THE APPENDED DRAWINGS

FIG. 1 illustrates an exploded schematic diagram of the aerosol-shielding endoscopy mouthpiece of the invention.

FIG. 2 illustrates a schematic diagram of the examinee wearing the aerosol-shielding endoscopy mouthpiece of the invention.

FIG. 3 illustrates a schematic diagram of the endoscope passing through the cuts on the cover to enter and exit the orifice.

FIG. 4A to FIG. 4D illustrate schematic diagrams of the cover showing different shapes.

### DETAILED DESCRIPTION OF THE INVENTION

A preferred embodiment of the invention is an aerosol-shielding endoscopy mouthpiece. In this embodiment, the aerosol-shielding endoscopy mouthpiece includes a bite block part for passing the endoscope and a mouth and nose-shielding part. In addition to allowing safe passage of the endoscope and breathing of the examinee, the bite block part includes a cover. The cover is disposed on the orifice of the bite block part and the cover has at least one cut for the endoscope to enter and exit and for shielding the gap between the endoscope and the edge of the orifice, so as to reduce the spread of aerosols and droplet ejected from the orifice during the examination process. The mouth and nose-shielding part is used for shielding the mouth and the nose of the examinee, so as to block body fluids and aerosols from the mouth and the nose of the examinee, and reduce the contamination during removal and processing.

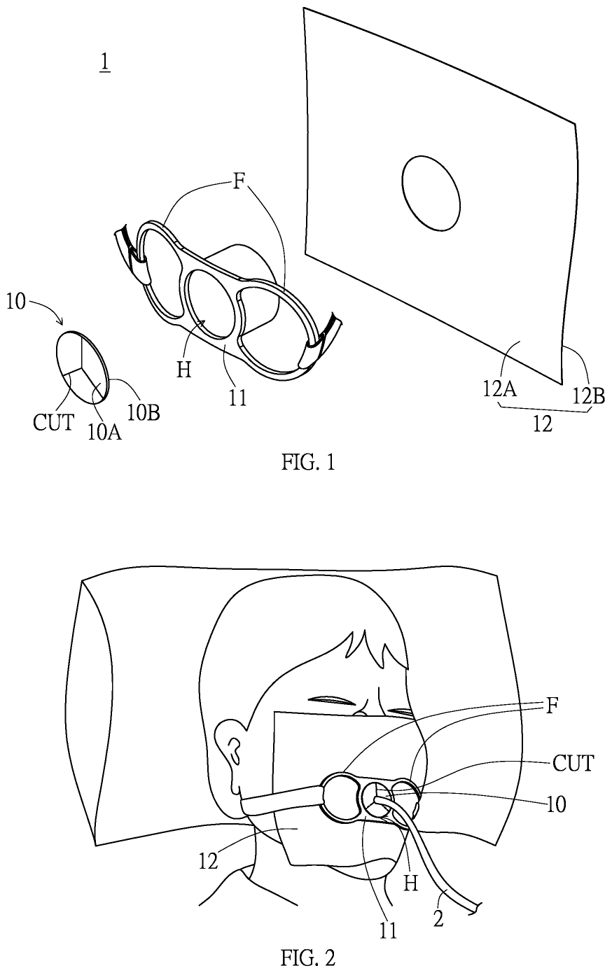

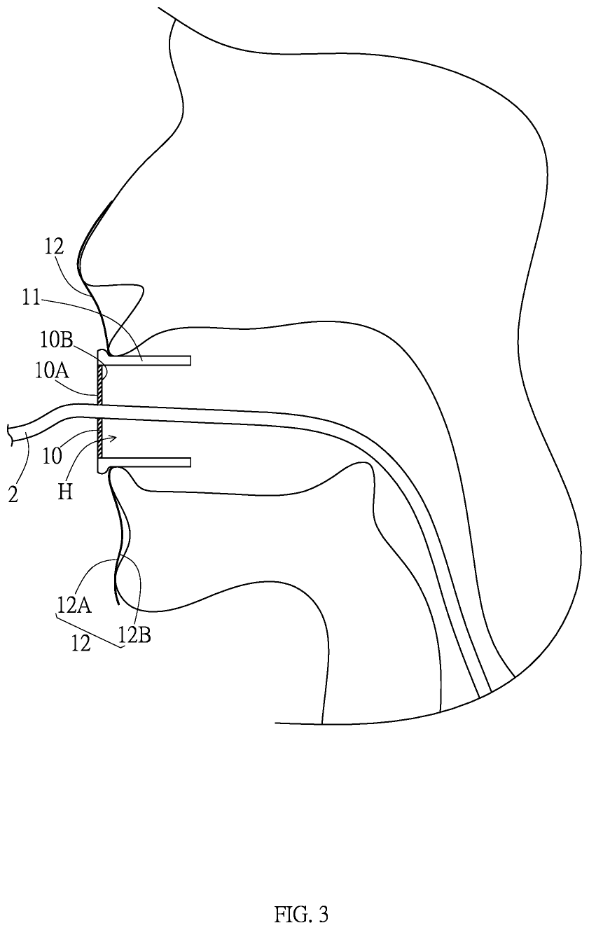

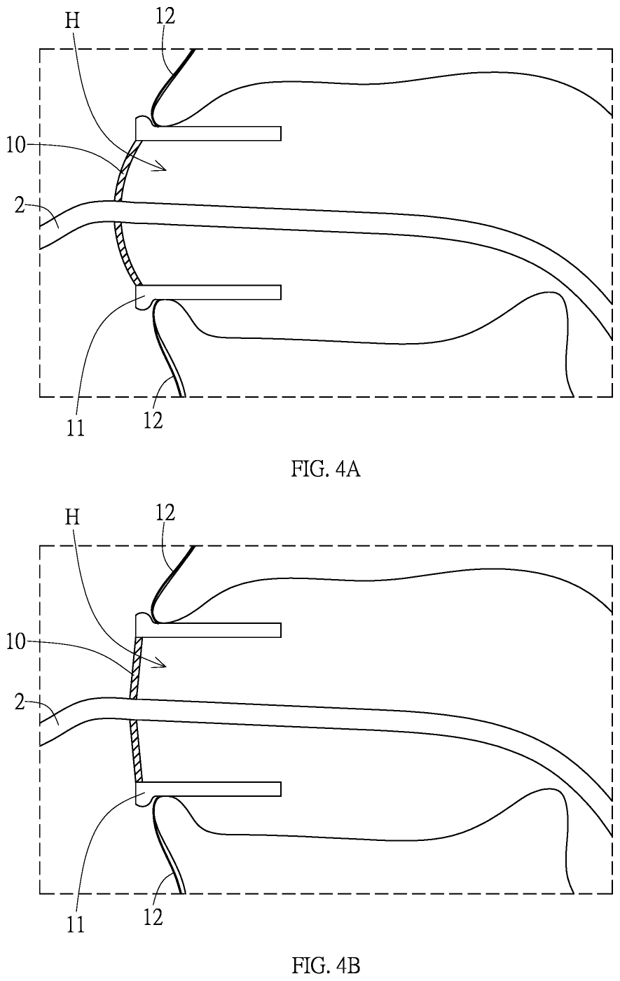

Please refer to FIG. 1 to FIG. 3. FIG. 1 illustrates an exploded schematic diagram of the aerosol-shielding endoscopy mouthpiece of the invention. FIG. 2 illustrates a schematic diagram of the examinee wearing the aerosol-shielding endoscopy mouthpiece of the invention. FIG. 3 illustrates a schematic diagram of the endoscope passing through the cuts on the cover to enter and exit the orifice.

As shown in FIG. 1 to FIG. 3, an aerosol-shielding endoscopy mouthpiece 1 includes a bite block part 11 for passing an endoscope 2 and a mouth and nose-shielding part 12. In addition to allowing safe passage of the endoscope 2 and breathing of an examinee, the bite block part 11 includes a cover 10. The cover 10 is disposed on an orifice H and the cover 10 has at least one cut CUT for the endoscope 2 to enter and exit and can also effectively shield a gap between the endoscope 2 and an edge of the orifice H so as to reduce the spread of aerosols and droplet ejected from the orifice H during the examination process. A fixing structure F is disposed on two sides of the bite block part 11. The fixing structure F can cooperate with an elastic band or other fixation around the examinee's head to fix the bite block part 11 to prevent the bite block part 11 from falling off or shifting during the examination process. The mouth and nose-shielding part 12 is used for shielding the mouth and the nose of the examinee, so as to block body fluids and aerosols from the mouth and the nose of the examinee, and reduce the contamination during removal and processing.

The mouth and nose-shielding part 12 has a first side 12A facing outside and a second side 12B facing the oral cavity, and the first side 12A and the second side 12B are made of different materials respectively. For example, the first side 12A facing outside can be made of any water-impermeable material to prevent its surfaces from being contaminated by the body fluids of the examinee; the second side 12B facing the oral cavity can be made of any highly absorbent material to effectively absorb the droplets and aerosols sprayed by the examinee. In addition, the first side 10A and the second side 10B of the cover 10 can be also designed in a way similar to the first side 12A and the second side 12B of the mouth and nose-shielding part 12, but not limited to this. In this way, the risk of infection of medical personnel should be greatly reduced, which can effectively improve the drawbacks of the prior arts.

In practical applications, the number, length and shape of the cut CUT on the cover 10 are not particularly limited, as long as the endoscope 2 can pass through the orifice H smoothly. An adhesive can be disposed on an edge of the mouth and nose-shielding part 12 and the adhesive can adhere to the face of the examinee to prevent splashing and liquid seepage from the edge of the mouth and nose-shielding part 12.

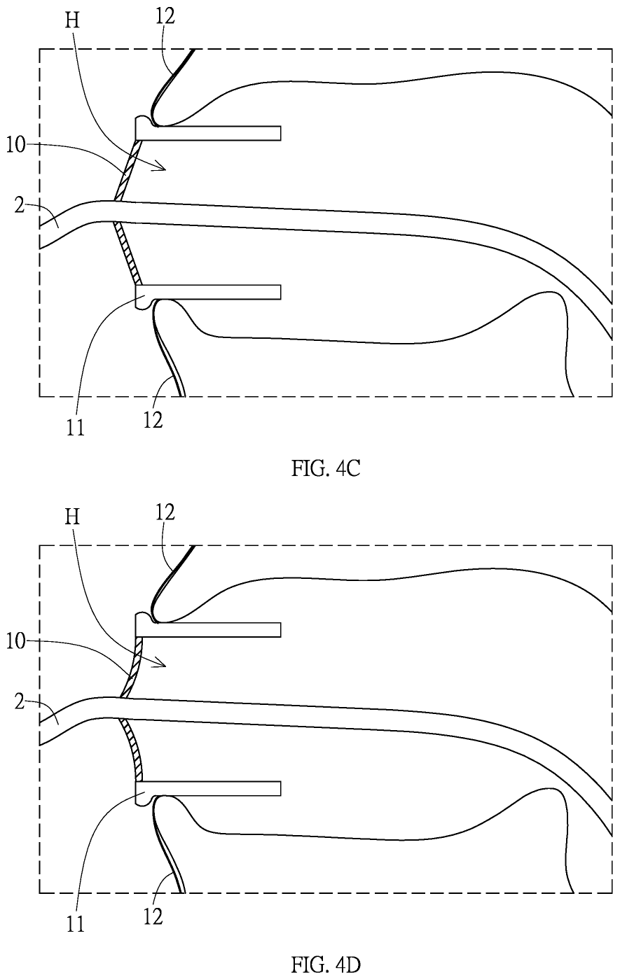

In addition, when the endoscope 2 passes through the cut CUT on the cover 10 to enter and exit the orifice H, the cover 10 can present different shapes for shielding the gap between the endoscope 2 and the edge of the hole H, such as the shapes of Oxygen mask, umbrella, trumpet or funnel, flat, etc., as shown in FIG. 4A to FIG. 4D.

Compared to the prior art, the aerosol-shielding endoscopy mouthpiece of the invention can be applied to all endoscopic examinations conducted through the oral cavity, thereby achieving the following specific effects: allowing the safe passage of the endoscope and the breathing of the examinee, reducing the spread of aerosols and droplet ejected from the orifice for the endoscope to pass through during the examination process, blocking body fluids and aerosols from the nose and the mouth of the examinee and reducing contamination during removal and processing; therefore, it can effectively reduce potential contamination and transmission risks to improve the drawbacks of the prior arts.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An aerosol-shielding endoscopy mouthpiece, comprising:
a mouth and nose-shielding part, for shielding a mouth and a nose of an examinee; and
a bite block part, comprising:
an orifice, for an endoscope to pass through; and
a cover, configured on the orifice and having at least one cut for the endoscope to pass through and shielding a gap between the endoscope and an edge of the orifice.

2. The aerosol-shielding endoscopy mouthpiece of claim 1, wherein during examination, the cover reduces aerosols and droplets from the orifice.

3. The aerosol-shielding endoscopy mouthpiece of claim 1, wherein the mouth and nose-shielding part blocks bodily fluids and aerosols from the nose and the mouth of the examinee and reduces contamination during removal and handling.

4. The aerosol-shielding endoscopy mouthpiece of claim 1, wherein the mouth and nose-shielding part has a first side facing outside and a second side facing oral cavity, and the first side and the second side are made of different materials.

5. The aerosol-shielding endoscopy mouthpiece of claim 4, wherein the first side is made of a water-impermeable material and the second side is made of a highly absorbent material.

6. The aerosol-shielding endoscopy mouthpiece of claim 1, wherein an adhesive is disposed on an edge of the mouth and nose-shielding part for adhering to a face of the examinee to prevent splashing and liquid seepage.

7. The aerosol-shielding endoscopy mouthpiece of claim 1, wherein a fixing structure is disposed on two sides of the bite block part, and the fixing structure can cooperate with an elastic band or other fixation around the examinee's head to fix the bite block part to prevent the bite block part from falling off or shifting during the examination process.
